(19) United States
(12) Patent Application Publication
(10) Pub. No.: US 2013/0133702 A1
(43) Pub. Date: May 30, 2013

(54) Mobile Spray Apparatus

(71) Applicants: John H. Reid, Fredericksburg, VA (US); Shane Reid, Fredericksburg, VA (US)

(72) Inventors: John H. Reid, Fredericksburg, VA (US); Shane Reid, Fredericksburg, VA (US)

(21) Appl. No.: 13/307,853

(22) Filed: Nov. 30, 2011

(51) Int. Cl. B08B 5/00; B05B 1/24; B08B 3/02

(52) U.S. Cl. 134/30; 239/135

(57) ABSTRACT

Spray apparatus including an air heater and mixer for mixing a liquid, typically a disinfectant or a cleaning agent, into the air stream created by the air heater. The spray apparatus can be provided on a carrier back frame or on a cart. A valve allows to regulate the amount of liquid dispersed into the air stream

### FIELD OF THE DISCLOSURE

An apparatus and a method for cleaning and disinfecting surfaces by providing a cleaning agent and/or a disinfectant solution mixed into a stream of pressurized air and/or water, which may optionally be heated. The apparatus can be carried in a back carrier or provided on a cart.

### BACKGROUND

Heavily frequented public areas, in various commercial, institutional, federal and private restrooms, locker rooms, and infant changing stations, pose unique challenges with regard to maintaining sanitary conditions. In some cases, for example restrooms at interstate highway rest stops, operations run 24 hours a day/365 days a year, without any prolonged periods in which the restrooms are closed for thorough cleaning.

Accordingly, it is desirable to provide the cleaning personal with equipment for cleaning and sanitizing public areas that allow for reducing the length of time in which the public area has to be closed or that even allow to keep the public area open during the cleaning process. Thus, it is desirable that the area to be cleaned can be quickly dried and returned to normal operation after the cleaning has been performed or that the cleaning can be performed without disturbance of the normal operation. Moreover, it desirable to increase the total area of, for example, a restroom, that can be cleaned by an employee within a given period of time.

In addition, attempts to control budgets by minimizing the expenditure for disinfectants, and the desire to curtail the environmental impact of these disinfectants by using only a minimum amount thereof, provides for an incentive for looking at new ways for cleaning and disinfecting heavily frequented public areas.

Moreover, it is desirable for a cleaning apparatus to be easily transportable and that consumables, such as detergents, can be easily refilled or replaced by new filled canisters.

However, conventional cleaning apparatuses are not satisfactory with regard to at least one of the aspects discussed above.

### SUMMARY OF THE DISCLOSURE

Disclosed herein is a spray apparatus comprising a mixing unit for mixing an air flow and a liquid flow; an air compressor being connected to an air intake port of the mixing unit; a heater for heating air, the heater being connected to the air compressor or being comprised in the air compressor; a canister for containing a liquid, the canister being connected to a liquid intake port of the mixing unit; and a valve for controlling an amount of the liquid being dispersed.

The spray apparatus allows for cleaning surfaces of, for example, toilets, sinks, and floors, with a small amount of cleaning agent or disinfectant solution possibly containing water, while adequately sanitizing these surfaces.

Further, a method for cleaning a surface utilizing the spray apparatus set forth above is disclosed, wherein the method comprises spraying the liquid onto the surface with the valve in an open position; closing the valve continuing to run the air compressor with the liquid valve closed to use the heated air flow by quickly drying the cleaned surface; or running the air compressor in a reverse direction; and drawing in at least a part of the dispersed liquid through the return flow.

### BRIEF DESCRIPTION OF THE DRAWINGS

FIG. 1 shows a schematic representation of the spray apparatus 100. Compartment 110 contains a liquid canister 120 for storing a cleaning agent/disinfectant, a motor 130 powering air compressor 140, an air intake 150, and a mixing unit 160. The air compressor 140 draws air through air intake 150, which optionally comprises an air intake filter (not shown). Air is mixed with a disinfectant and/or sanitizing agent in mixing unit 160 and guided through a flexible hose 180 to spray nozzle 195. The operator sets operational parameters on control unit 190, which is described in more detail in FIGS. 4a and 4b.

FIG. 2 shows a flow diagram of the fluids of the spray apparatus. An air flow 210 is taken in through air intake 150 and compressed in air compressor 140. Further, the air is heated in air heater 280 provided within air compressor 140. The heated air 210 is mixed in mixing unit 160 with a liquid cleaning agent stored in liquid canister 120. The mixture 230 is guided through flexible hose 180 to spray nozzle 195 and dispersed in spray pattern 240. Valve 170 can be closed by the operator such that the fluid 230 leaving the mixing unit consists only of heated and compressed air and does not contain the cleaning agent. In one embodiment, the air compressor can be run in reverse, such that liquid is taken up through spray nozzle 195 and removed as fluid 250 through mixing unit 160, passed on to the air compressor as fluid 260, and evaporated as fluid 270.

FIG. 3 shows a spray apparatus in which compartment 110 is provided in a back carrier. Motor 130 is powered by battery pack 310 and/or by power chord 320.

FIG. 4a shows a top view of control unit 190. Button 410, which is provided on both sides of control unit 190 to allow right and left hand use, opens and closes valve 170. Indicator 420 specifies the power remaining in battery pack 310. Similarly, indicator 430 specifies the amount of liquid remaining in liquid canister 120. Knobs 440, 450, and 460 adjust the temperature of the compressed air, the air pressure, and the amount of liquid mixed into mixing unit 160. Mixture 230 is supplied to spray nozzle 195 by flowing in the direction indicated by arrow 470.

FIG. 4b shows a side view of control unit 190. Trigger 480 engages the air compressor.

FIG. 5a shows an embodiment of the spray apparatus in which compartment 110 is provided in a four-wheeled cart.

FIG. 5b shows an embodiment of the spray apparatus in which compartment 110 is provided in a two-wheeled cart. Retractable leg 510 allows for use of the spray apparatus without the need of manually holding the cart in an upright position.

FIGS. 6a and 6b show spray nozzle 195 equipped with brush 620.

FIGS. 7a and 7b show spray nozzle 195 equipped with squeegee 710.

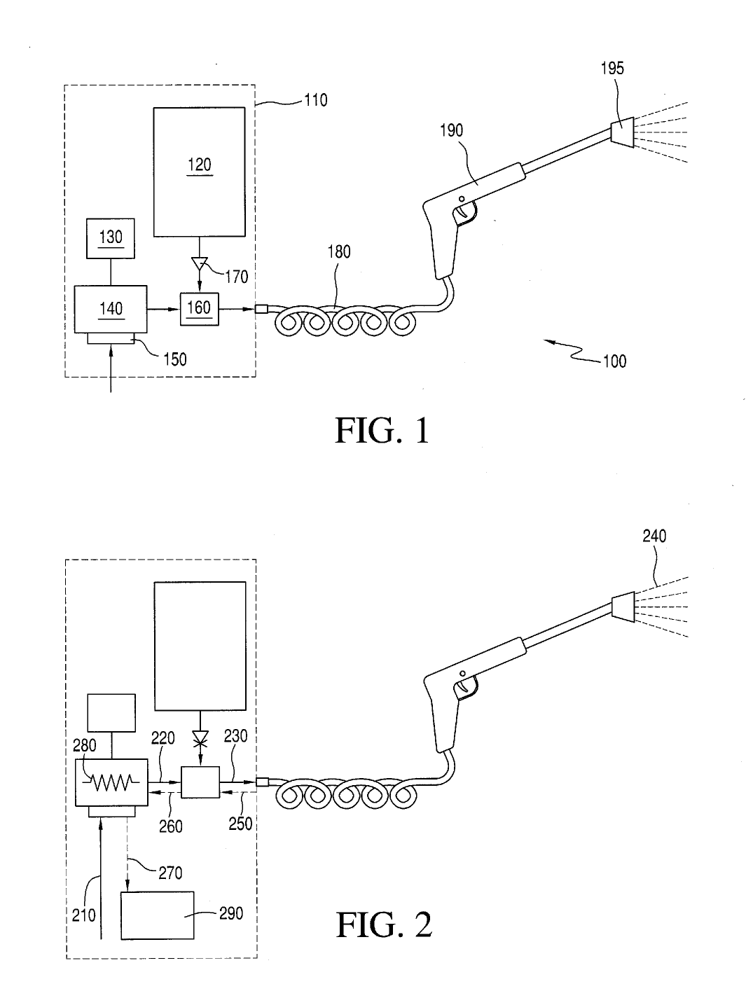

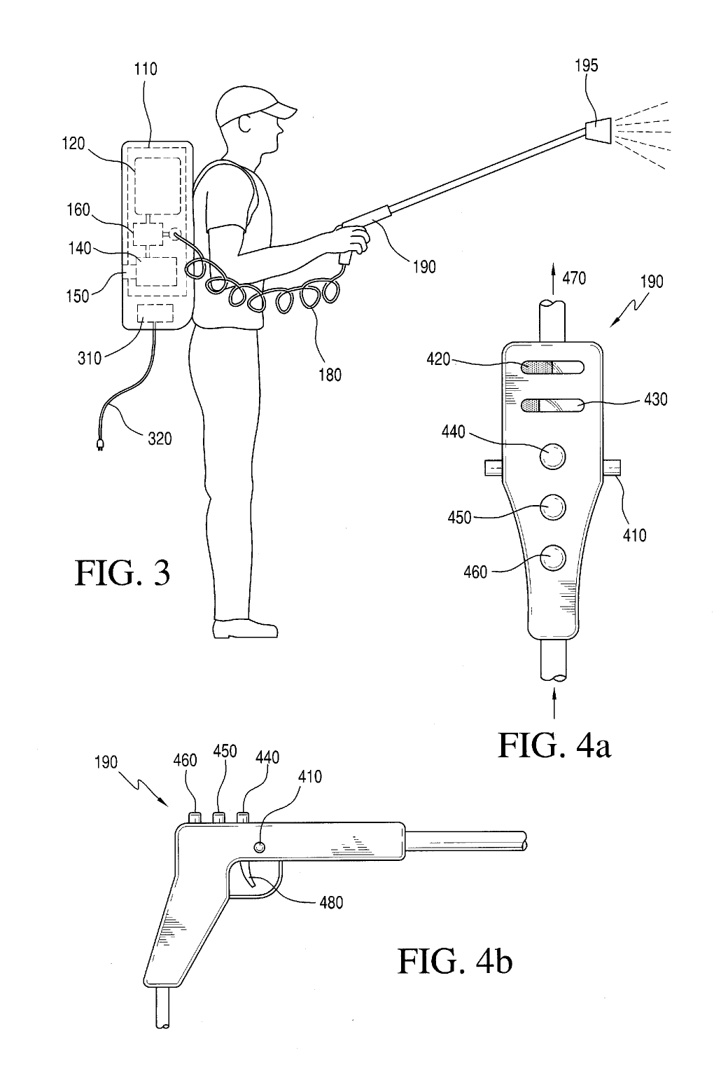

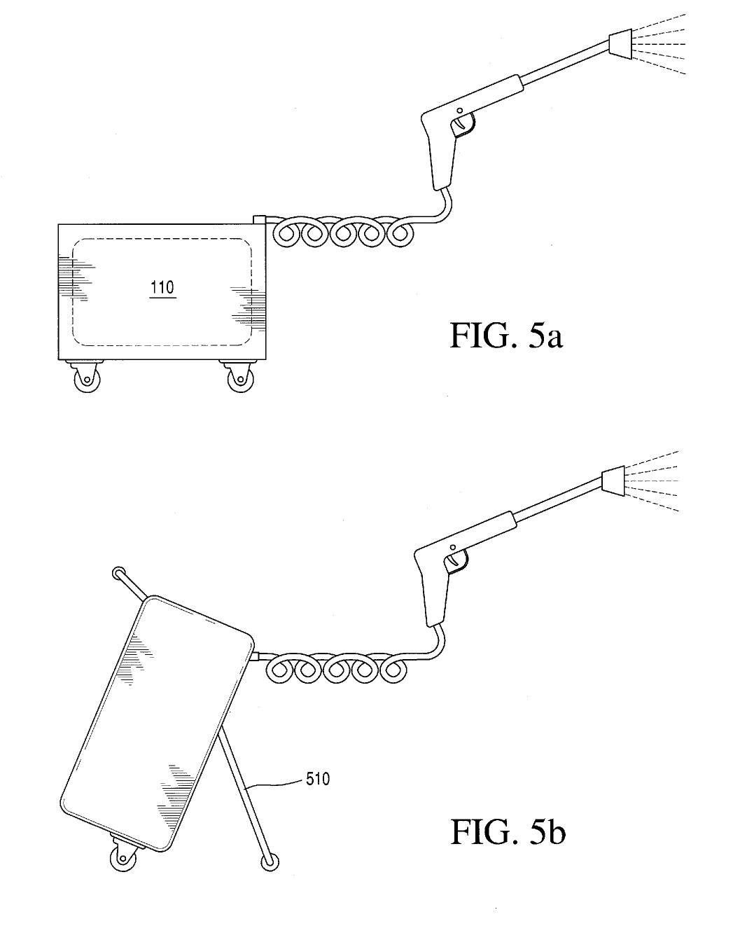

### DESCRIPTION OF THE BEST AND VARIOUS EMBODIMENTS

The foregoing and other objects, aspects, and advantages will be better understood from the following detailed description of the best and various embodiments. Throughout the various views and illustrative embodiments of the present disclosure, like reference numbers are used to designate like elements.

In a typical embodiment, the mixing unit 160 is an eductor comprising a converging-diverging nozzle to convert the pressure energy of the compressed air 220 to velocity energy, which creates a low pressure zone that draws in and entrains the liquid from liquid canister 120. After passing through the throat of the eductor, the mixed air and liquid expand and the velocity is reduced which results in recompressing the mixed fluids by converting velocity energy back into pressure energy.

In another typical embodiment, the liquid from the liquid canister 120 is injected into the air stream mixing unit with a chemical pump.

The liquid comprises a disinfectant solution, a cleaning agent solution, or a mixture thereof, which may include water. Other solvents, such as an alcohol, in particular ethanol, acetone, and ethyl acetate are also envisioned, however, the preferred solvent is water. The disinfectant destroys microorganisms that are living on the surface to be cleaned, whereas the cleaning agent cleans the surface by removing residues from the surface. Unless explicitly stated otherwise, the liquid mixed with the airflow is hereinafter referred to as the cleaning agent for simplicity, even though the liquid may contain a cleaning agent, a disinfectant, or a cleaning agent and a disinfectant. Further, the cleaning agent may contain additional additives, such as, but not limited to, surfactants, preservatives, and fragrances. Typically, commercially available cleaning agents are used.

The spray apparatus further comprises an adjustable spray nozzle 195 for selecting a spray profile. The spray profile can be adjusted to cover a large area, for spot cleaning, or an intermediate setting. In a particular embodiment, the spray nozzle can pivot to provide for different spraying angles.

In a particular embodiment, the spray apparatus further comprises a holder for removably attaching a cleaning tool. With particularity, the cleaning tool is selected from the group consisting of a squeegee and a brush. One or more cleaning tools may be attached simultaneously.

Status data, such as battery level indicated by indicator 420 or canister fill level indicated by indicator 430 are communicated between control unit 190 and compartment 110. Likewise, operational settings such as air temperature, air pressure, and amount of liquid added are set by the operator and communicated between control unit 190 and compartment 110. In a particular embodiment, control unit 190 communicates with the components of compartment 110 through electrical or optical data cables running alongside or incorporated into flexible hose 180. In another particular embodiment, control unit 190 and compartment 110 communicate through wireless transmission in the radio frequency range.

In a typical embodiment, brush 610 having bristles 620 is reversibly attached to spray head 195 using a screw-mount. However, brush 610 may also be attached using a magnet, in particular a rare earth magnet, a clamp, or a spring-loaded retaining mechanism. In another embodiment, exchangeable nozzles may be provided with various cleaning tools, such that a particular cleaning tool may be selected with a nozzle having a spray profile adapted for the cleaning tool. Further, the ability to quickly replace a spray nozzle allows the operator to continue the cleaning process without prolonged interruption in case the openings in the spray nozzle become blocked.

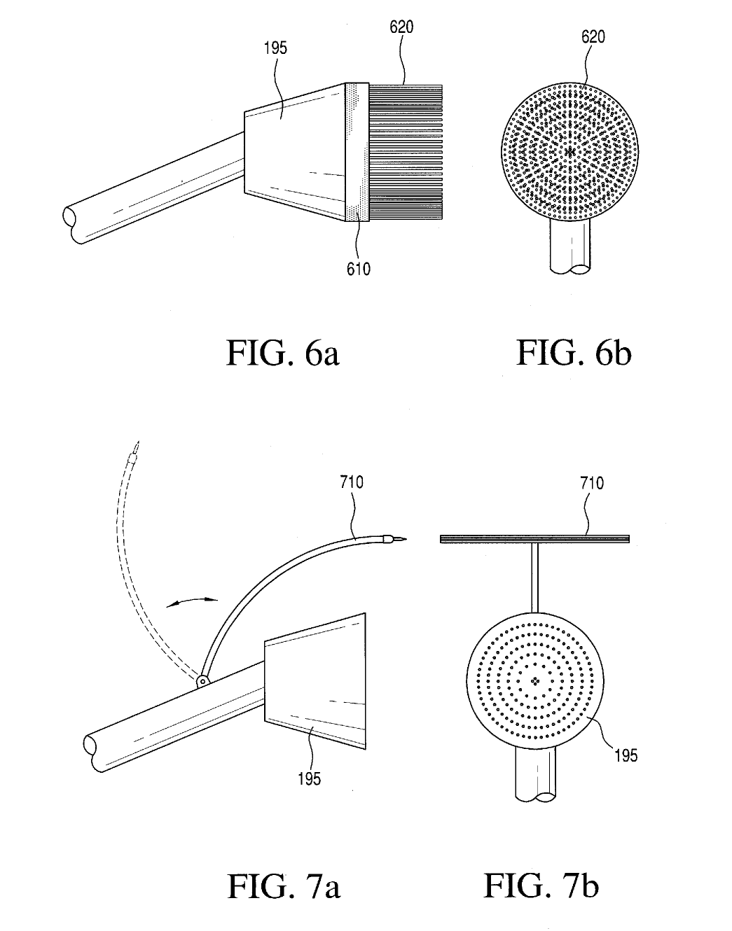

FIG. 6b shows bristles 620 provided in concentric rings in-between openings in the spray nozzle 195 that are also provided as concentric rings. In this arrangement, mixture 230 is at its highest temperature outside the spray apparatus when it jointly with bristles 620 removes deposits from the surface to be cleaned.

The air compressor is typically powered by an electrical motor. However, the air compressor may also be powered by clean burning fuels, such as propane.

In a typical embodiment, the spray apparatus comprises an electrical power cord. In another typical embodiment, the spray apparatus comprises a battery pack. In yet another typical embodiment, the electrical motor is powered using the electrical power cord while the battery pack is recharged.

In a particular embodiment, compartment 110 and, if present, battery pack 310 are provided in a back carrier frame. The back carrier frame is carried by the operator using shoulder straps leaving both hands for cleaning operations. Optionally, a chest strap and/or a hip strap may be provided to prevent the back carrier frame from slipping.

In another particular embodiment, the spray apparatus is mounted on a wheel cart. The cart is typically a four-wheeled cart. Also typically, the cart is a two-wheeled cart.

With particularity, liquid canister 120 is removable to allow for a quick swap with a full canister if the cleaning agent has been depleted during the cleaning process. Further, the ability to quickly swap the canister allows to change the cleaning agent for a particular purpose, such as when surfaces requiring a more aggressive cleaning agents have to be treated.

Typically, the liquid canister is refillable. Refilling may occur with the canister removed or mounted in compartment 110. In a particular embodiment, the canister may only be opened by qualified operators using a specialized tool to avoid that chemically incompatible cleaning agents are mixed during the refilling.

In a particular embodiment, a return flow is provided for a part of the liquid being dispersed, wherein the compressor is a reversible compressor to draw in excess liquid formed on the surface to be cleaned. Accordingly, a build-up of material dispersed in the excess liquid, which would otherwise accumulate on the surface, can be avoided.

In another particular embodiment, a second canister 290 is provided for collecting the dispersed liquid from the return flow. Alternatively, an impermeable membrane may be provided within liquid canister 120 and the return flow channeled to liquid canister 120 such that the dispersed liquid is collected within liquid canister 120, but separated from unused cleaning agent by the impermeable membrane.

Typically, the heater heats the air to a temperature of from 30° C. to 100° C., a temperature of from 50° C. to 80° C. being more typical. Also typically, the air compressor compresses the air to a pressure of from 1.1 bar (16 psi) to 10 bar (145 psi), a pressure of from 5 bar (72.5 psi) to 8 bar (116 psi) being more typical.

In a typical embodiment, a method for cleaning comprises returning the air compressor to a default direction and drying the surface with a heated air flow.

The embodiments described hereinabove are further intended to explain best modes known of practicing the disclosure and to enable others skilled in the art to utilize the disclosure in such, or other, embodiments and with the various modifications required by the particular applications or uses. Accordingly, the description is not intended to limit the disclosure to the form disclosed herein. Also, it is intended that the appended claims be construed to include alternative embodiments.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

What is claimed is:

1. A spray apparatus, comprising:
a mixing unit for mixing an air flow and a liquid flow;
an air compressor being connected to an air intake port of the mixing unit;
a heater for heating air, the heater being connected to the air compressor or being comprised in the air compressor; or the compressed air is heated by the compressor.
a canister for containing a liquid, the canister being connected to a liquid intake port of the mixing unit; and
a valve for controlling an amount of the liquid being dispersed.

2. The spray apparatus according to claim 1, wherein the mixing unit is an eductor.

3. The spray apparatus according to claim 1, wherein the mixing unit is a chemical pump.

4. The spray apparatus according to claim 1, wherein the liquid comprises a disinfectant, a cleaning agent, or a mixture thereof.

5. The spray apparatus according to claim 1, further comprising an adjustable spray nozzle for selecting a spray profile.

6. The spray apparatus according to claim 1, further comprising a holder for removably attaching a cleaning tool.

7. The spray apparatus according to claim 6, wherein the cleaning tool is selected from the group consisting of a squeegee and a brush.

8. The spray apparatus according to claim 1, wherein the air compressor is powered by an electrical motor.

9. The spray apparatus according to claim 8, further comprising an electrical power cord.

10. The spray apparatus according to claim 8, further comprising a battery pack.

11. The spray apparatus according to claim 1, further comprising a back carrier frame.

12. The spray apparatus according to claim 1, being mounted on a wheel cart.

13. The spray apparatus according to claim 1, wherein the canister is removable.

14. The spray apparatus according to claim 1, wherein the canister is refillable.

15. The spray apparatus according to claim 1, further comprising a return flow for a part of the liquid being dispersed, wherein the compressor is a reversible compressor.

16. The spray apparatus according to claim 15, further comprising a second canister for collecting the dispersed liquid from the return flow.

17. The spray apparatus according to claim 1, wherein the heater heats the air to a temperature of from 30° C. to 100° C.

18. The spray apparatus according to claim 1, wherein the air compressor compresses the air to a pressure of from 1.1 bar to 10 bar.

19. A method for cleaning a surface, comprising:
providing the spray apparatus according to claim 1,
spraying the liquid onto the surface with the valve in an open position;
closing the valve; and
drying the surface with a heated air flow.

20. A method for cleaning a surface, comprising:
providing the spray apparatus according to claim 15,
spraying the liquid onto the surface with the valve in an open position;
closing the valve;
running the air compressor in a reverse direction; and
drawing in at least a part of the dispersed liquid through the return flow.

21. The method according to claim 20, further comprising
returning the air compressor to a default direction; and
drying the surface with a heated air flow.
